(19) United States
(12) Patent Application Publication
(10) Pub. No.: US 2016/0169871 A1
(43) Pub. Date: Jun. 16, 2016

(54) METHOD FOR EVALUATING EFFICACY OF A PERSONAL CARE PRODUCT

(71) Applicant: Conopco, Inc., d/b/a UNILEVER, Englewood Cliffs, NJ (US)

(72) Inventors: Li LIN, Ningbo City (CN); Jing ZHANG, Shanghai (CN)

(73) Assignee: Conopco, Inc., d/b/a UNILEVER, Englewood Cliffs, NJ (US)

(21) Appl. No.: 14/904,454

(22) PCT Filed: Jun. 27, 2014
(86) PCT No.: PCT/EP2014/063702

§ 371 Date: Jan. 12, 2016

(30) Foreign Application Priority Data
Jul. 19, 2013 (CN) PCT/CN2013/079730
Sep. 9, 2013 (EP) 13183501.9

(51) Int. Cl. G01N 33/50

(52) U.S. Cl. CPC G01N 33/5097; G01N 2500/10

(57) ABSTRACT

A method for evaluating the hydration efficacy of a personal care product or a component thereof, the method comprising: selecting a first portion of fresh plant tissue; treating the first portion of plant tissue with the personal care product or the component thereof; and heating the treated first portion of plant tissue to a drying temperature of from 35 to 80° C.

### TECHNICAL FIELD OF THE INVENTION

The present invention relates to a method for evaluating the hydration efficacy of a personal care product or a component thereof. In particular the invention relates to measurement of the hydrating efficacy of personal care products, such as skin or hair care products, using a method which results in more rapid and immediate assessment.

### BACKGROUND TO THE INVENTION

Personal care products which improve the condition of the skin or hair are often designed to do so by influencing the ability of the skin or hair to absorb and/or retain moisture. Unfortunately, however, the benefits of such products may not be immediately perceived by a user and in fact may take many hours or even days of repeat application to yield consumer-perceivable benefits.

In vivo and in vitro instrumental measurements of the hydration state of tissue are often complicated and/or involve expensive laboratory equipment. Furthermore such measurements often result in numerical parameters that are difficult for laymen to understand or at least relate to the expected product efficacy.

The present inventors have thus identified a need to provide methods which can demonstrate the hydrating ability of cosmetics but which does not need specialist equipment and/or is easily related to consumer benefits.

US patent published as U.S. Pat. No. 4,373,382 (Nicole A. A. Brun) discloses a method of ascertaining the hydrating action of a product to be applied on the skin wherein the product to be tested is applied to at least one portion of the surface of a first vegetable sample, for example an apple, and this first sample is stored together with at least another untreated sample of the same vegetable, under the same storage conditions, and the surface evolution of these samples is compared.

Whilst the method of U.S. Pat. No. 4,373,382 does not necessarily require complex instrumentation and effectively demonstrates consumer benefits through the change in condition of a vegetable substrate, the method is still time consuming with days or weeks needed to show significant effects. As such, the method of U.S. Pat. No. 4,373,382 is ill suited to situations where a rapid or even immediate demonstration of product efficacy is required. Such situations include for example, in-store or point-of-sale demonstrations.

The present inventors have now found that some or all of the abovementioned disadvantages may be ameliorated by the provision of a specific method.

### SUMMARY OF THE INVENTION

In a first aspect the present invention provides a method for evaluating the hydration efficacy of a personal care product or a component thereof, the method comprising:
(i) selecting a first portion of fresh plant tissue;
(ii) treating the first portion of plant tissue with the personal care product or the component thereof; and
(iii) heating the treated first portion of plant tissue to a drying temperature of from 35 to 80° C.

Fresh plant tissue is found to be a moisture-sensitive substrate and is often visually compelling. Furthermore by utilizing a drying temperature of from 35 to 80° C., rapid and visually-apparent changes in plant tissue can be induced by drying without heat-induced damage such as burning, scorching, blistering, stewing or otherwise cooking.

All other aspects of the present invention will more readily become apparent upon considering the detailed description and examples which follow.

### DETAILED DESCRIPTION

The method of the present invention comprises a step (i) of selecting a first portion of fresh plant tissue. The plant tissue may be an entire plant, flowers, leaves, petals, stems, fruits, seeds or roots, and/or the fragments thereof. Most preferred are petal, leaf or a combination thereof, as these are typically relatively thin but with a large surface area and so dry relatively quickly. Most preferably the plant tissue is petal or a fragment thereof as petals are typically more fragile than leaves and so give more visually apparent changes on drying.

By “fresh” plant tissue is meant tissue that has never been dried to a water content of less than 30% by weight, and usually has a water content in the range 60 to 95%. The method of the present invention preferably utilizes fresh plant tissue within 24 hours of removal of the tissue (e.g. by picking, pruning and/or cutting) from a living plant, more preferably within 12 hours, more preferably still within 6 hours and most preferably within 10 s to 3 hours.

To allow for especially rapid drying it is preferred that the first portion is not too large. In particular it is preferred that the first portion has a mass of less than 10 g, more preferably less than 2 g, more preferably still less than 1 g, even more preferably less than 0.5 g and most preferably less than 0.2 g.

The first portion should not be too small, however. In particular the first portion should be visible to the naked eye. Preferably the first portion has a mass of at least 1 mg, more preferably at least 10 mg and most preferably at least 50 mg.

The method of the present invention comprises a step (ii) of treating the first portion of plant tissue with the personal care product or the component thereof.

The personal care product is preferably one intended for application to the hair and/or skin for the purpose of improving the condition thereof. In particular the product is preferably intended to improve a condition of hair and/or skin selected from hydration, barrier function, moisture retention and combinations thereof. Preferably the product is a hair care product, especially a hair care product intended to improve the condition of the scalp.

The treatment comprises at least contacting the first portion with the product or component. The contact may, for example, comprise spreading the product or component on at least one surface of the portion. Additionally or alternatively the contact may comprise soaking the portion in a liquid comprising the product or component. The treatment may also comprise rinsing the portion following contact with the product or component. A rinsing step is especially preferred if the product is a rinse-off product such as a skin cleanser, body wash, shampoo or hair conditioner.

In a preferred embodiment the first portion is treated with a component of the personal care product in step (ii). Treating the portion with the component rather than the entire product allows, for example, the component to be applied to the portion in a higher concentration than in the product and/or to be applied in a different manner than would be achieved by applying the whole product. Thus the hydrating efficacy of the component can be enhanced such that the same may be demonstrated in a short time. Preferably the first portion of plant tissue is treated with an aqueous liquid comprising the component. More preferably the concentration of the component by weight of the aqueous liquid is greater than the concentration of the component by weight of the personal care product. For example the concentration of the component by weight of the aqueous liquid may be at least twice, more preferably at least three times, more preferably still at least five times and most preferably at least ten times the concentration of the component by weight of the personal care product.

The component should be associated with the product in some manner. By “associated” is meant that the method preferably comprises a step of identifying the component as an ingredient of the product. For example, prior to step (i) the method may comprise a step of selecting a component of the personal care product for assessment. Additionally or alternatively, the method may comprise a step of communicating the component as an ingredient of the personal care product through indicia such as text, video, audio and the like.

The component should be selected to be a component with good hydration efficacy. For example the component preferably is or at least comprises a humectant, emollient, bioactive or combination thereof. Most preferably the component comprises or is polyhydric alcohol, fatty materials (such as oils, fatty alcohols, fatty acids and/or soaps), ester emollient, hydrocarbon emollient, silicone oil, vitamin, plant extract, or a mixture thereof.

Preferred polyhydric alcohols include polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, isoprene glycol, 1,2,6-hexanetriol, glycerol, ethoxylated glycerol, propoxylated glycerol and mixtures thereof. Most preferred is glycerol (also known as glycerin).

Silicone oils may be divided into the volatile and nonvolatile variety. The term “volatile” as used herein refers to those materials which have a measurable vapor pressure at ambient temperature (25° C.). Volatile silicone oils are preferably chosen from cyclic (cyclomethicone) or linear polydimethylsiloxanes containing from 3 to 9, preferably from 4 to 5, silicon atoms.

Nonvolatile silicone oils useful as an emollient material include polyalkyl siloxanes, polyalkylaryl siloxanes and polyether siloxane copolymers. The essentially nonvolatile polyalkyl siloxanes useful herein include, for example, polydimethyl siloxanes with viscosities of from about 5×10⁻⁶ to 0.1 m²/s at 25° C. Among the preferred nonvolatile emollients useful in the present compositions are the polydimethyl siloxanes having viscosities from about 1×10^{-s} to about 4×10⁻⁴ m²/s at 25° C.

Organopolysiloxane crosspolymers can be usefully employed. Representative of these materials are dimethicone/vinyl dimethicone crosspolymers and dimethicone crosspolymers available from a variety of suppliers including Dow Coming (9040, 9041, 9045, 9506 and 9509), General Electric (SFE 839), Shin Etsu (KSG-15, 16 and 18 [dimethicone/phenyl vinyl dimethicone crosspolymer]), and Grant Industries (Gransil brand of materials), and lauryl dimethicone/vinyl dimethicone crosspolymers supplied by Shin Etsu (e.g. KSG-31, KSG-32, KSG-41, KSG-42, KSG-43 and KSG-44).

Specific examples of fatty materials include stearyl alcohol, glyceryl monoricinoleate, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, eicosanyl alcohol, behenyl alcohol, cetyl palmitate, di-n-butyl sebacate, isopropyl myristate, isopropyl palmitate, isopropyl stearate, butyl stearate, polyethylene glycol, triethylene glycol, lanolin, cocoa butter, com oil, cotton seed oil, olive oil, palm kernel oil, rape seed oil, safflower seed oil, evening primrose oil, soybean oil, sunflower seed oil, avocado oil, sesame seed oil, coconut oil, arachis oil, castor oil, acetylated lanolin alcohols, petroleum jelly, mineral oil, butyl myristate, isostearic acid, palmitic acid, isopropyl linoleate, lauryl lactate, myristyl lactate, decyl oleate, myristyl myristate, and mixtures thereof.

Among the ester emollients are:
a) Alkenyl or alkyl esters of fatty acids having 10 to 20 carbon atoms. Examples thereof include isoarachidyl neopentanoate, isodecyl neopentanoate, isononyl isonanoate, cetyl ricinoleate, oleyl myristate, oleyl stearate, and oleyl oleate.
b) Ether-esters such as fatty acid esters of ethoxylated fatty alcohols.
c) Polyhydric alcohol esters. Butylene glycol, ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl mono-stearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters. Particularly useful are pentaerythritol, trimethylolpropane and neopentyl glycol esters of C₁-C₃₀ alcohols. Exemplative is pentaerythrityl tetraethyl hexanoate.
d) Wax esters such as beeswax, spermaceti wax and tribehenin wax.
e) Sterols esters, of which cholesterol fatty acid esters are examples thereof.
f) Sugar ester of fatty acids such as sucrose polybehenate and sucrose polycottonseedate.

Hydrocarbons which are useful include petrolatum, mineral oil, C₁₁-C₁₃ isoparaffins, polyalphaolefins, and especially isohexadecane.

The duration of the treatment step (ii), i.e., the time between starting to apply the personal care product or component to the plant tissue and commencement of the heating step (iii) is preferably between 1 s and 24 hours. However, the method may be especially suitable for situations where rapid demonstration of product efficacy is desired, such as for example, in-store and/or at point of sale. Thus it is preferred that the duration of the treatment step is less than 2 hours, more preferably less than 1 hour, more preferably still less than 30 minutes and most preferably less than 10 minutes.

The method of the present invention comprises a step (iii) of heating the treated first portion of plant tissue to a drying temperature of from 35 to 80° C.

To provide even more rapid drying it is preferred that the drying temperature is at least 40° C., most preferably at least 45° C.

To ensure that the rapid and visually-apparent changes in plant tissue can be induced by drying without heat-induced damage, it is preferred that the plant tissue is not held at too high a temperature for too long a time. Thus it is preferred that the duration of the heating step, i.e., the time at which the temperature of the plant tissue is at the drying temperature, is between 10 s and 1 hour, more preferably between 30 s and 40 minutes, more preferably still between 1 and 20 minutes, and most preferably between 2 and 10 minutes. Additionally or alternatively the drying temperature is less than 75° C., more preferably less than 70° C., most preferably less than 60° C.

The heating may be effected in any convenient manner. For example the heating step may comprise contacting the treated plant tissue with a stream of hot air, for example from a hair dryer. Additionally or alternatively the heating step may comprise incubating the treated plant tissue in an oven.

Following step (iii) the method preferably comprises a step (iv) of assessing at least one attribute of the treated first portion. Most preferably the attribute assessed is appearance, such as size, shape, surface texture, colour or a combination thereof. Additionally or alternatively mechanical properties such as brittleness, fragility etc. may be assessed.

The method of the present invention is particular effective when used to evaluate the hydration efficacy of the personal care product or the component relative to a placebo. Thus in a most preferred embodiment the method comprises selecting a second portion of fresh plant tissue in step (i); treating the second portion with a placebo product in step (ii); and treating the second treated portion to the heating step (iii).

The second portion should be substantially identical to the first portion, for example in respect of the type of plant material as well as shape and size.

The placebo may be any composition different from the personal care product or component to be tested. However most preferably the placebo is water (or at least comprises at least 99% water by weight of the placebo product, more preferably 99.9 to 100%).

The assessed attribute in step (iv) is preferably a change in a characteristic of the treated first portion relative to fresh plant tissue and/or relative to the treated second portion. The characteristic is preferably appearance and/or mechanical properties.

In one embodiment the method comprises a step of capturing one or more images of the plant material and storing and/or transmitting the one or more images. The image(s) may, for example, be stored on a recordable medium such as CD, flash drive or other computer-readable memory. The image(s) may be transmitted, for example, for display on one or more visible display units. Suitable visible display units include, for example, monitors, TV screens and/or mobile device screens.

Except in the examples, or where otherwise explicitly indicated, all numbers in this description indicating amounts of material or conditions of reaction, physical properties of materials and/or use may optionally be understood as modified by the word “about”.

All amounts are by weight of the product, unless otherwise specified.

It should be noted that in specifying any range of values, any particular upper value can be associated with any particular lower value.

For the avoidance of doubt, the word “comprising” is intended to mean “including” but not necessarily “consisting of” or “composed of”. In other words, the listed steps or options need not be exhaustive.

The disclosure of the invention as found herein is to be considered to cover all embodiments as found in the claims as being multiply dependent upon each other irrespective of the fact that claims may be found without multiple dependency or redundancy.

The following examples are provided to facilitate an understanding of the invention. The examples are not intended to limit the scope of the claims.

### EXAMPLES

### Example 1

A test sample was prepared by mixing 7.5 g of CLEAR® shampoo, 7.5 g CLEAR® hair conditioner and 35 g of water. The shampoo comprised 0.05% w/w glycerine and 0.01% w/w cactus extract. The conditioner comprised 0.1% glycerine and 0.01% w/w cactus extract.

The placebo was 50 g of water.

Four petals were taken from an evening primrose (oenothera rosea) plant. Two were placed in the test sample and two were placed in the placebo. After soaking for 5 minutes the petals were removed from the test sample and placebo and each rinsed under running water for 5 s.

The rinsed petals were spread apart on a plate and placed in an oven preheated to 50° C. After 15 minutes the petals were removed from the oven and examined visually.

It was observed that the petals treated with the test sample still looked like fresh petals and showed little sign of dehydration. In contrast, the petals treated with the placebo showed visible signs of dehydration including shrinkage, surface wrinkles and deepening of colour.

### Example 2

To demonstrate the efficacy of some of the components of the hair care products described in Example 1, a test sample was prepared by mixing 7.5 g of glycerine, 1.5 g cactus extract and 41 g of water.

The placebo was 50 g of water.

Four petals were taken from an evening primrose (oenothera rosea) plant. Two were placed in the test sample and two were placed in the placebo. After soaking for 5 minutes the petals were removed from the test sample and placebo. The petals were allowed to drain for a few seconds but no rinsing step was performed.

The treated petals were spread apart on a plate and placed in an oven preheated to 50° C. After 15 minutes the petals were removed from the oven and examined visually.

It was observed that the petals treated with the test sample still looked like fresh petals and showed little sign of dehydration. In contrast, the petals treated with the placebo showed visible signs of dehydration including shrinkage, surface wrinkles and deepening of colour.

1. A method for evaluating the hydration efficacy of a personal care product or a component thereof, the method comprising:
(i) selecting a first portion of fresh plant tissue;
(ii) treating the first portion of plant tissue with the personal care product or the component thereof; and
(iii) heating the treated first portion of plant tissue to a drying temperature of from 35 to 80° C.
wherein a second portion of fresh plant tissue is selected in step (i); the second portion is treated with a placebo product in step (ii); and the second treated portion is also treated to the heating step (iii);
and further wherein following step (iii) the method comprises a step (iv) of assessing at least one attribute of the treated first portion wherein the assessed attribute is a change in a characteristic of the treated first portion relative to fresh plant tissue and/or relative to the treated second portion; and
wherein the duration of the heating step (ii) is between 10 s and 1 hour.

2. The method as claimed in claim 1, wherein the placebo is water.

3. The method as claimed in claim 1, wherein the characteristic is appearance and/or mechanical properties.

4. The method as claimed in claim 1, wherein the plant tissue comprises petal, leaf or a combination thereof, preferably petal.

5. The method accord to claim 1, wherein in step (ii) the first portion of plant tissue is treated with an aqueous liquid comprising the component.

6. The method as claimed in claim 5, wherein the concentration of the component by weight of the aqueous liquid is greater than the concentration of the component by weight of the personal care product.

7. The method as claimed in claim 1, wherein the duration of the heating step (ii) is between 1 and 20 minutes.

8. The method as claimed in claim 1, wherein the heating step (iii) comprises contacting the treated plant tissue with a stream of hot air.

9. The method as claimed in claim 1, wherein the drying temperature is less than 70° C.

10. The method as claimed in claim 9, wherein the drying temperature is in the range 40 to 70° C.

11. The method as claimed in claim 1, wherein the product is a hair care product.

12. The method as claimed in claim 1, wherein the component is selected from polyhydric alcohol, fatty material, ester emollient, hydrocarbon emollient, silicone oil, vitamin, plant extract, or a mixture thereof.
